SEQUENCE LISTING (8 sequences)

SEQ ID NO: 1 - DNA, 54 bp, Artificial
  Oligonucleotide primer
gagctcggtaccgtcgacaagggccctgcacggcgagattttcaggagctaagg

SEQ ID NO: 2 - DNA, 54 bp, Artificial
  oligonucleotide primer
tactaaaagcttgtcgacaagggccctgcacttacgccccgccctgccactcat

SEQ ID NO: 3 - DNA, 54 bp, Artificial
  oligonucleotide primer
gagctcggtaccgtcgacaagggccctcaagggcgagattttcaggagctaagg

SEQ ID NO: 4 - DNA, 54 bp, Artificial
  oligonucleotide primer
tactaaaagcttgtcgacaagggccctcaagttacgccccgccctgccactcat

SEQ ID NO: 5 - DNA, 54 bp, Artificial
  oligonucleotide primer
gagctcggtaccgtcgacaagggccctccagggcgagattttcaggagctaagg

SEQ ID NO: 6 - DNA, 54 bp, Artificial
  oligonucleotide primer
tactaaaagcttgtcgacaagggccctccagttacgccccgccctgccactcat

SEQ ID NO: 7 - DNA, 54 bp, Artificial
  oligonucleotide primer
gagctcggtaccgtcgacaagggcccttcagggcgagattttcaggagctaagg

SEQ ID NO: 8 - DNA, 54 bp, Artificial
  oligonucleotide primer
tactaaaagcttgtcgacaagggcccttcagttacgccccgccctgccactcat

(19) United States
(12) Patent Application Publication
(10) Pub. No.: US 2006/0166334 A1
(43) Pub. Date: Jul. 27, 2006

(54) Method and compositions for rapidly modifying clones

(75) Inventor: Shuwei Yang, Germantown, MD (US)

Correspondence Address: BAKER DONELSON BEARMAN CALDWELL & BERKOWITZ, PC, 555 11TH STREET, NW, 6TH FLOOR, WASHINGTON, DC, 20004, US

(73) Assignee: GENECOPOEIA, INC., Germantown, MD (US)

(21) Appl. No.: 11/312,459

(22) Filed: Dec. 21, 2005

Related U.S. Application Data
(60) Provisional application No. 60/637,461, filed on Dec. 21, 2004.

(51) Int. Cl. C12P 19/34; C12N 15/74

(52) U.S. Cl. 435/91.2; 435/471

(57) ABSTRACT

A method for removing extraneous nucleotides in a cloned coding sequence using a type IIs endonuclease, the method comprising introducing a linker that comprises at least one recognition site for a Type IIs restriction endonuclease to the ORF, cloning the ORF into a suitable vector, and removing the extraneous nucleotides from the vector with a RE IIs digestion. Also provided are a vector, a kit and oligonucloetides suitable for the invention.

### FIELD OF INVENTION

This invention relates to the use of type IIs restriction enzymes (RE IIs) for rapidly modifying or manipulating nucleic acid molecules, such as genes cloned in expression vectors. Specifically, this invention relates to compositions and methods of using Type IIs restriction enzymes, optionally in combination with the polymerase chain reaction (PCR) or “Rec-join” cloning methodologies, for removing stop or start codons in a vector containing an open reading frame (ORF) of interest that is to be expressed.

### BACKGROUND OF THE INVENTION

Site-specific recombination-based cloning, alone or in combination with a more traditional ligation step, has been widely adopted in constructing ORF clone collections. Highly efficient site-specific recombination-based systems are available from commercial suppliers. For example, “Rec-Join” cloning methods are described, for example, in U.S. patent application Ser. No. 10/627,711 (Publication No. 20040115812), the entire content of which is incorporated herein by reference.

These cloning technologies have lead to increased availability of completed genome and cDNA sequences for many organisms. For example, sequence-verified, full-length human cDNA clones, probably covering almost all of the human genome, are available from a number of sources, including commercially.

Researchers interested in determining the functions of these genes desire to have these clones in a format that can be easily expressed. However, for the vast majority of available ORF clones, the ORF sequence of interest, or other “desired sequence segment,” is flanked by extraneous nucleotides or bases for restriction enzyme recognition sites or recombination sites engineered to facilitate the cloning efforts. These extraneous nucleotides interfere with subsequent expression efforts or protein functional studies, for example altering the primary or tertiary structural or functional characteristics of the protein, or contain stop codons that prevent translation of downstream sequences that encode expression or purification tags. These ORF clones, as a consequence, have to be re-cloned and re-sequenced in order for them to be expressed and studied at the protein or other functional level, adding tremendous burdens to the researchers in terms of cost and time.

There is a need for methods that allow seamless transfer of cloned ORFs into a suitable expression vector, while removing the undesirable flanking nucleotides, without the need of re-cloning and re-sequencing.

### DESCRIPTION OF THE INVENTION

The present invention provides methods and compositions that take advantage of the properties of type IIs restriction enzymes (RE IIs), for rapidly and reliably modifying nucleic acid molecules, such as removing the extraneous flanking nucleotides of a cloned ORF. Specifically, this invention provides compositions and methods of using Type IIs restriction enzymes, optionally in combination with the polymerase chain reaction (PCR) or “Rec-join” cloning methodologies and digestion with regular type II endonucleases, for removing unwanted flanking sequences, such as stop codons in a vector containing an open reading frame (ORF) of interest that is to be expressed.

Based on their characteristics, endonucleases are grouped, in general, into three major types or classes: I, II (including IIs) and III. Class I enzymes cut somewhat randomly, and Class III are rare and are not pertinent to this invention. Most enzymes used in molecular biology are type II enzymes. These enzymes often are called restriction enzymes because they recognize a particular target sequence (i.e., restriction endonuclease recognition site) and break the polynucleotide chains within or near to the recognition site. A type II recognition sequence may be continuous or interrupted.

A subgroup of type II restriction enzymes (RE II) are called Class IIs enzymes (i.e., type IIs enzymes, or RE IIs). RE IIs have asymmetric recognition sequences, and cleavage occurs at a distance wary from the recognition site (for a review, see Szybalski et al. Gene 100:13-26 (1991)).

Unlike other RE II enzymes, RE IIs endonucleases generally recognize non-palindromic sequences and cleave outside of their recognition site. U.S. Pat. No. 4,293,652 discloses a linker with a type-IIs enzyme recognition sequence to permit synthesized DNA to be inserted into a vector without disturbing a recognition sequence. Brousseau et al. (Gene 17:279-289 (1982)) and Urdea et al. (Proc. Natl. Acad. Sci. USA 80:7461-7465 (1983)) disclose the use of type-IIs enzymes for the production of vectors to produce recombinant insulin and epidermal growth factor respectively.

In one embodiment, the present invention provides a method for modifying a first polynucleotide molecule that comprises a desired segment, such as an ORF, and extraneous nucleotides at either or both of the 5′ and 3′ ends of the desired segment, the method comprising 1) engineering the first polynucleotide molecule to contain an extraneous nucleotide removal linker (ENRL), or engineering a second polynucleotide molecule to contain an ENRL, wherein the ENRL comprises at least one recognition site for a Type IIs restriction endonuclease (RE IIs Site), 2) joining the first polynucleotide molecule with the second polynucleotide molecule to form a third polynucleotide molecule which comprises the desired segment, the extraneous nucleotides and at least one RE IIs site, and 3) digesting the third polynucleotide molecule with the RE IIs, wherein at least some of the extraneous nucleotides are removed.

In the context of the present invention, an extraneous nucleotide removal linker (ENRL) may also be referred to as an extraneous base removal linker (EBRL) or a base removal linker. It is designed or constructed such that after a cleavage by a RE IIs, some or all of the extraneous nucleotides or bases flanking the nucleotide sequence of interest, such as an ORF, are removed. An EBRL suitable for the present invention itself or in combination with other sequences of the molecule of which it is a part, should contain a RE IIs recognition site and cleavage site.

It is important to recognize that if the removed extra nucleotide space are located between that encoding N-terminal and/or C-terminal Tag and ORF, the BRL need to be engineered such that after the removal of the extraneous nucleotides, the N-terminal Tag, the ORF of interest, and C-terminal Tag coding sequence, if present, remain in frame for translation purposes, and no additional stop codons should be created.

Many RE IIs are known and available to those of ordinary skills in the art. A partial list of them include AarI, Acc36I, AceIII, AclWI, AloI, AlwI, Alw26I, AlwXI, AsuHPI, BaeI, BaeI, Bbr7I, BbsI, BbvI, BbvII, Bbv16II, BccI, Bce83I, BceAI, BcefI, BcgI, BcgI, BciVI, Bco5I, Bco116I, BcoKI, BfiI, BfuI, BfuAI, BinI, Bli736I, Bme585I, BmrI, BmuI, BpiI, BpmI, BpuAI, BpuEI, BpuSI, BsaI, BsaXI, BsaXI, BscAI, BseMII, BseRI, BsgI, BsmAI, BsmFI, Bsp24I, Bsp24I, BspCNI, BspMI, BsrDI, BstF5I, BtgZI, BtsI, CjeI, CjeI, CjePI, CjePI, CspCI, CspCI, CstMI, EciI, Eco31I, Eco57I, Eco57MI, Esp3I, FalI, FalI, FauI, FokI, GsuI, HaeIV, HaeIV, HgaI, Hin4I, HphI, HpyAV, Ksp632I, MboII, Mlyl, MmeI, MnlI, PleI, PpiI, PpiI, PsrI, PsrI, RleAI, SapI, SfaNI, SspD5I, Sth132I, StsI, TaqII, TaqII, TspDTI, TspGWI, TstI, TstI, and Tth111II. The recognition and cleavage sites of these enzymes are also well-known. An ordinarily skilled person will easily recognize that the choice the RE IIs for a particular purpose of base removal is determined by the sequence of the ORF, the extraneous bases to be removed and other downstream or upstream sequences.

Type IIs restriction enzyme recognition sites and type IIs restriction enzymes that are useful in the present cloning methods for EBRL or SCRL in the second polynucleotide molecule, compositions, nucleic acids, vectors and kits include, but are not limited to, in the table 1, BsaI, BbsI, BbvII, BsmAI, BspMI, Eco31I, BsmBI, BaeI, FokI, HgaI, MlyI, SfaNI and Sth132I. The first, and second restriction sites of EBRL or SCRL in the second polynucleotide molecule, if present, utilized throughout the various aspects of the present invention may be the same or they may be different. In addition, the restriction sites on the same nucleic acid molecule (and/or nucleic acid segment) may be the same, or they may be different. The present invention also encompasses situations wherein one or both of the nucleic acid molecules involved in the various methods are vectors, and where one or both of the nucleic acid molecules are linear nucleic acid molecules. The present invention also encompasses the use of other blunt-end cleavage enzymes, including, but not limited to, ScaI, SmaI, HpaI, HincII, HaeIl and AluI. The present invention also encompasses the use of other sticky-end cleavage enzymes, including, but not limited to, EcoRI kpnI, Not I, Xho I. The present invention also encompasses the use of one site specific recombination attachment sites are, but not limited, are selected from the group consisting of attB sites, attP sites, attL sites, attR sites, lox sites, psi sites, tnpI sites, dif sites, cer sites, frt sites, and mutants, variants and derivatives thereof.

According to the present invention, an EBRL may be engineered to a primer that is used to amply an ORF of interest. Thus the present invention are directed to PCR primers that contain different Type II Restriction sites. These primers may include both 5′ and 3′ primers combined with other cloning joining sites (e.g. topoisomerase sites). The amplified product may in turn be transferred, into a suitable vector, via recombination cloning, or digestion and ligation using a regular RE II enzyme. Once cloned, the ORF will be adjacent to a EBRL, which, upon digestion with the appropriate RE IIs, will remove the extraneous bases and the ORF can be manipulated for further expression experiments, e.g. transferred into a suitable expression vector. Appropriate transformation steps or amplification/propagation steps may be included in the process, as is well recognized by those skilled in the art.

In a preferred embodiment, the first polynucleotide molecule comprises at its 3′ end a stop codon which is removed. This is often desired when down stream tag sequences are desired to be expressed, which tag sequences are needed for isolation or purification or other purposes. In another embodiment, 3′ non-translated sequences, or 5′ non-translated sequences are removed according to the present invention.

According to another preferred embodiment, the method of the present invention removes extraneous bases flanking an ORF what has already been cloned into a vector, such as those in “entry clones.” In this case, the second polynucleotide molecule is a vector comprising an ENRL, and the ORF in the entry clone is transferred, e.g. via recombination cloning, into the vector comprising the ENRL, which is similarly processed via RE IIs digestion and further expression manipulations. It is recognized that the first (entry clone) and second polynucleotide molecules (vector comprising ENRL) may also joined by a combination of at least two of site specific recombination, restriction digestion, and ligation.

As used herein, a “vector” is a nucleic acid molecule (preferably DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A Vector can have one or more restriction endonuclease recognition sites (whether type I, II or IIs) at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Vectors can also comprise one or more recombination sites that permit exchange of nucleic acid sequences between two nucleic acid molecules.

Methods of inserting a desired nucleic acid fragment which do not require the use of recombination, transpositions or restriction enzymes (such as, but not limited to, UDG cloning of PCR fragments. TA Cloning, PCR cloning (also known as direct ligation cloning), can also be applied to clone a fragment into a cloning vector to be used according to the present invention. The cloning vector can further contain one or more selectable markers suitable for use in the identification of cells transformed with the cloning vector.

The above can be used for many commercially available ORF clones with stop codons such as entry clones, via vector switch. It is recognized that at least one type II restriction site is needed for vector switch, and the location of Type II site is often upstream of type IIs site.

The expression vector comprising the ORF of interest, without the undesirable extraneous nucleotides, should comprise, operably linked to the ORF, one or more suitable transcription or translation regulatory elements. These elements are widely available and well-known to those skilled in the art, and may include promoters, enhancer elements, replication origins, and ribosome binding sites etc.

The expression vector method may further comprise at least one of a selection marker, an expression tag, and a purification tag.

In one preferred embodiment, the first polynucleotide molecule may comprise a first selection marker, and the second polynucleotide molecule may comprise a second selection marker. As a consequence, the third polynucleotide molecule may comprise both the first and second selection markers, such that the appropriate molecules may be readily selected. May selectable markers are known and available, such as antibiotic resistance genes, fluorescent markers, auxotrophic markers, toxic markers and phenotypic markers.

Specific antibiotic resistance genes include chloramphenicol resistance gene, ampicillin resistance gene, tetracycline resistance gene, Zeocin resistance gene, spectinomycin resistance gene and kanamycin resistance gene. Toxic markers may include a ccdB gene, a gene encoding a tus protein, a kicB gene, a sacB gene, an ASK1 gene, a φX174 E gene and a DpnI gene.

Alternatively, digestion with the RE IIs of the third polynucleotide molecule may removes one of the two selection markers, resulting in a fourth polynucleotide molecule comprising the ORF but only one of the first or second selection markers. This may help appropriate differentiation between the third and fourth molecules.

The present invention further provides a vector that comprises at least one ENRL, which includes at one RE IIs site. The vector may further comprise a tag sequence downstream of the ENRL, or a tag sequence upstream of the ENRL.

The present invention further provides a kit comprising the vector described above, and at lease one Type IIs restriction enzyme. Preferably, the kit further comprises at least one type II restriction enzyme. The kit may further comprise at least one DNA ligase, and a suitable buffer for one or more of the enzymes.

The present invention also provides oligonucleotide comprising an ENRL either in its 5′-end or 3′-end, or both, such as those specifically exemplified in the figures which are discussed in more detail below.

It is recognized that the ligation and restriction steps of the present inventive method can be altered depending on the vectors used. Furthermore, the digestion and ligation steps may be performed simultaneously if the restriction enzymes are chosen such that the reconstituted site after ligation cannot be cut by the original restriction enzymes (e.g. Xho I and Smal I). Many such sites are available and known to those skilled in the art.

### BRIEF DESCRIPTION OF THE DRAWINGS

FIG. 1a depicts one embodiment of the present invention, wherein a BRL (base removal linker) is added to the ORF of interested via PCR, which is combined with a vector with a BRL of its own. FIGS. 1b and 1c show a situation only the cloning vector (FIG. 1b) or the PCR product (FIG. 1c) contains a BRL. The two BRL may or may not be the same, and may or may not contain the same RE IIs site. When the PCR product and the vector is combined, the BRL is altered (e.g. due to digestion using a RE II enzyme and ligation, creating a BRL). In addition, in both FIGS. 1b and 1c, the vector is shown to have a suitable promoter, in which case the resulting vector can be converted to an expression vector by simply digesting with a RE IIs followed with a suitable ligation or joining step.

FIG. 2 depicts another embodiment of the present invention. The ORF of interest is transferred using methods well-known to those of skill in the art, from a first vector (Vector A) to another vector (Vector B). Vector B is engineered such that it contains at least one recognition site for a RE IIs. Transfer of the ORF from Vector A into Vector B produces a Vector B containing a stop codon from the ORF. Further digestion of the Vector B resulting in the removal of the undesired stop codon. Vector B may contain a promoter (FIG. 2B), in which case further digestion with the cognate RE IIs and a ligation step will produce a desired expression vector. Vector B may contain no promoter (FIG. 2A), in which case the ORF without the undersired stop codon needs to be transferred to another vector, using methods well-known in the art.

FIG. 3a shows a vector with a specific BRL sequence, with two BsgI sites in opposite directions. This arrangement facilitates the removal of the extraneous nucleotides, and the resulting overhang ends can be simply ligated. FIGS. 3b and 3c show yet another specific BRL sequence, and describe the specific steps of using two different combinations of RE II and RE IIs digestion and ligation for removal of the undesired stop codon and linking the coding sequence for the GFP (green fluorescent protein) in frame without creating additional stop codons.

FIG. 4a shows three different BRLs suitable for linking to a N-terminal tag sequence. FIGS. 4b-4d describe the specific steps of using different combinations of RE II and RE IIs digestion and ligation for removal of N-terminal extraneous nucleotides.

FIG. 5a shows the coding sequence of the Cm resistance gene. FIG. 5b lists primers used. FIG. 5c depicts the pDeliver clone. FIG. 5d describes the steps of constructing the Intermediate cloning vector. FIG. 5e shows the steps of removal of the stop codon and the construction of the final expression vector.

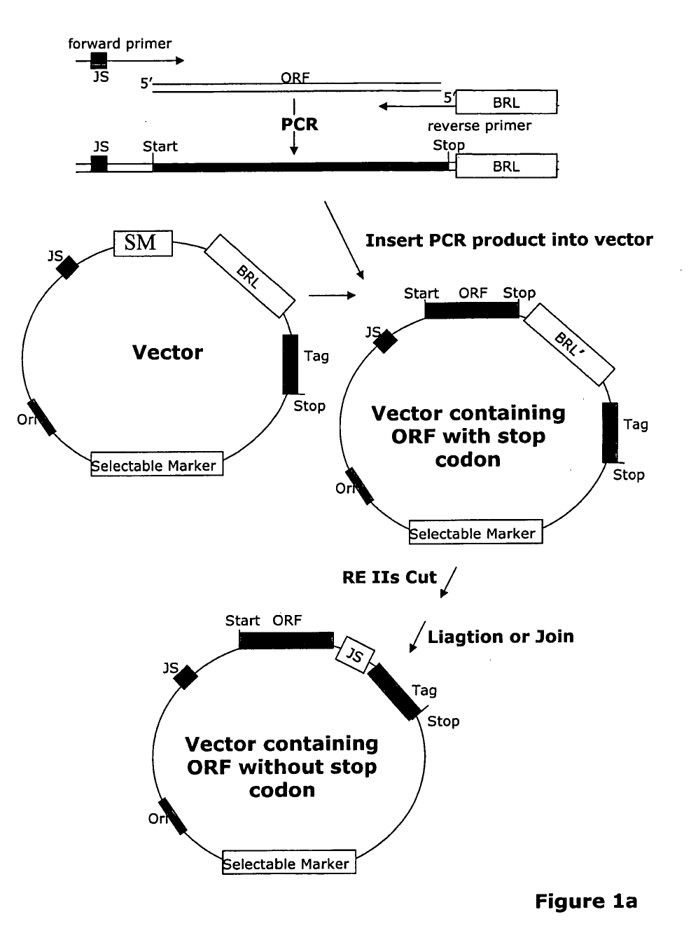

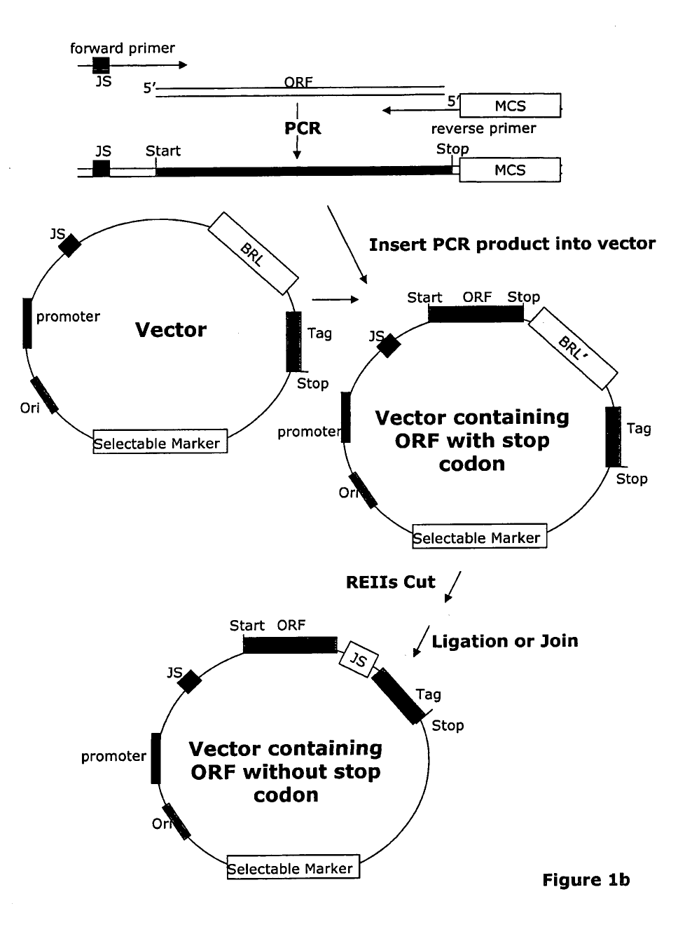

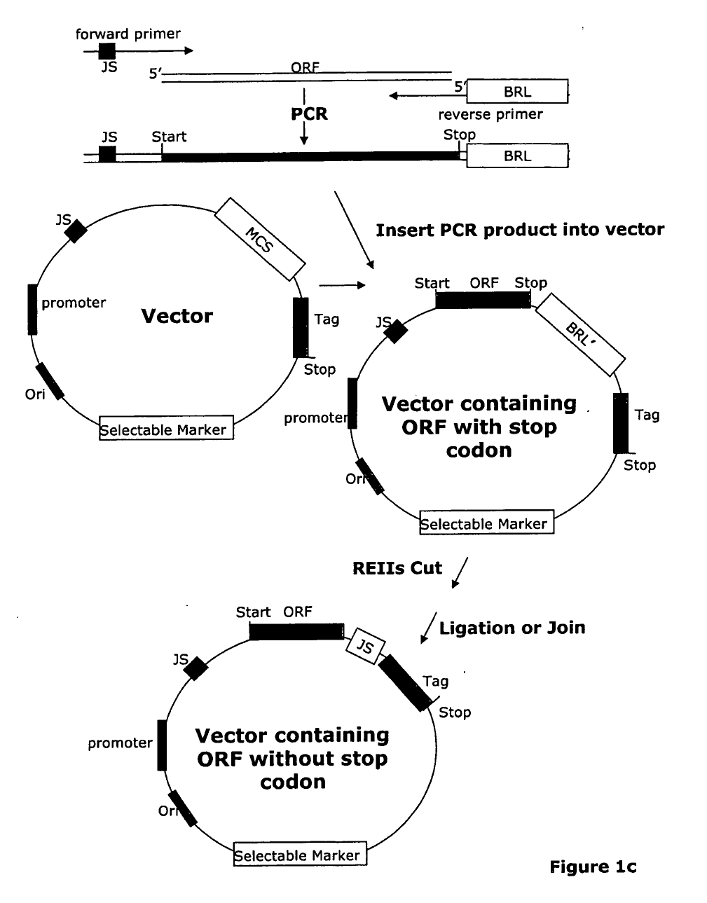

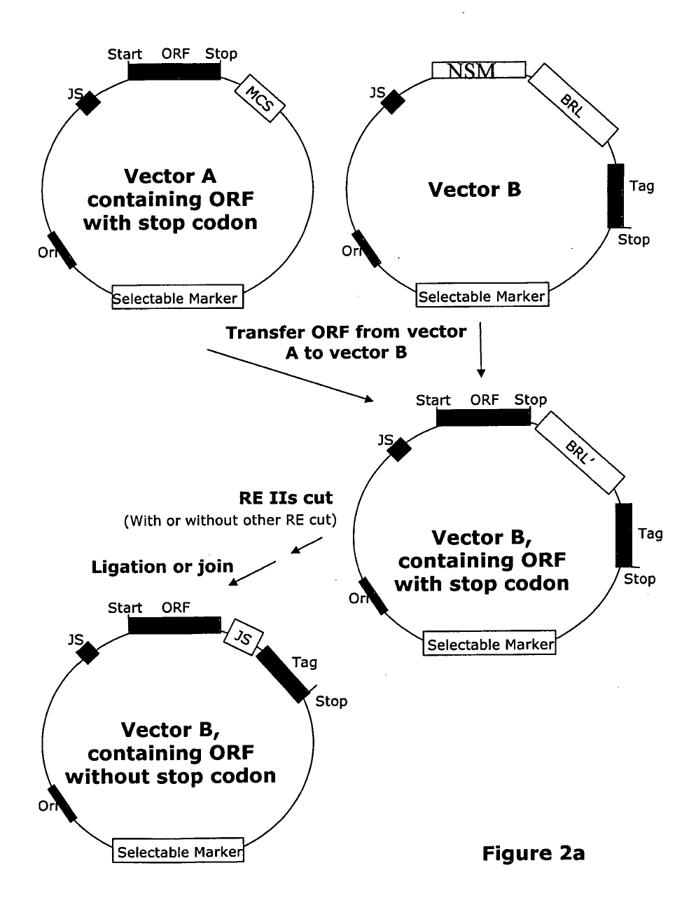

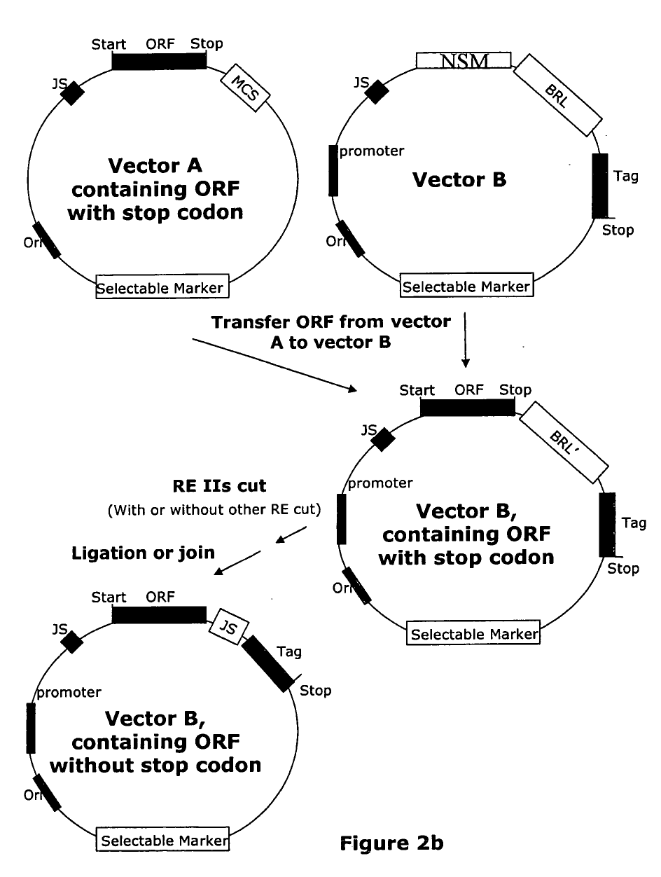

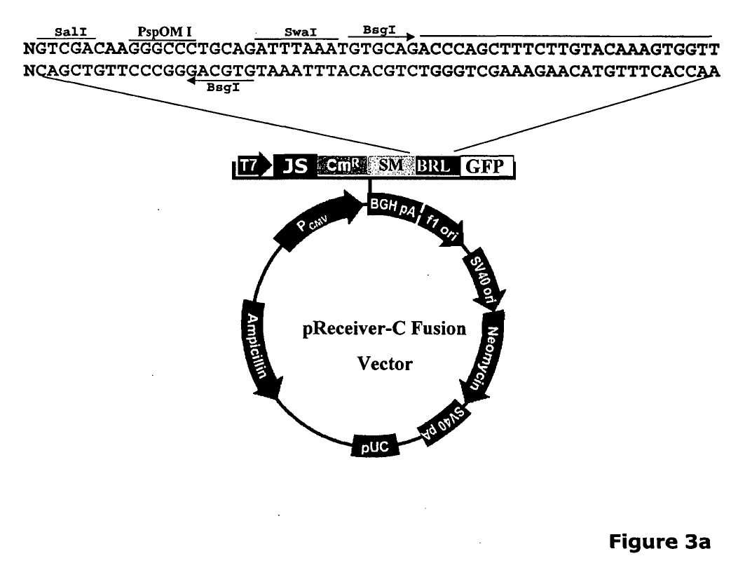

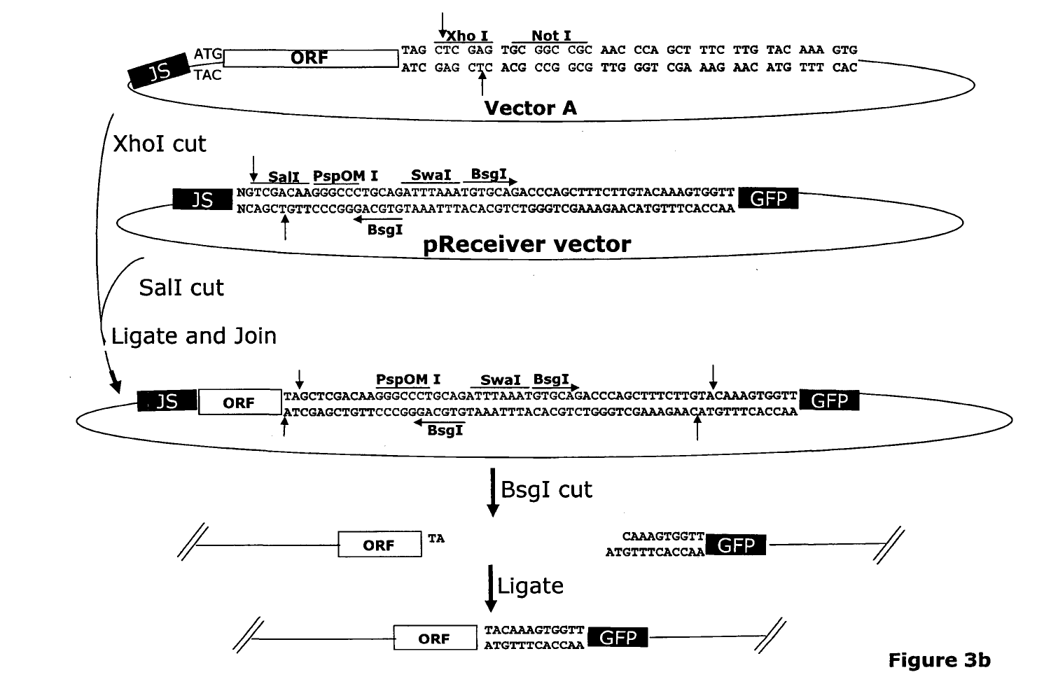

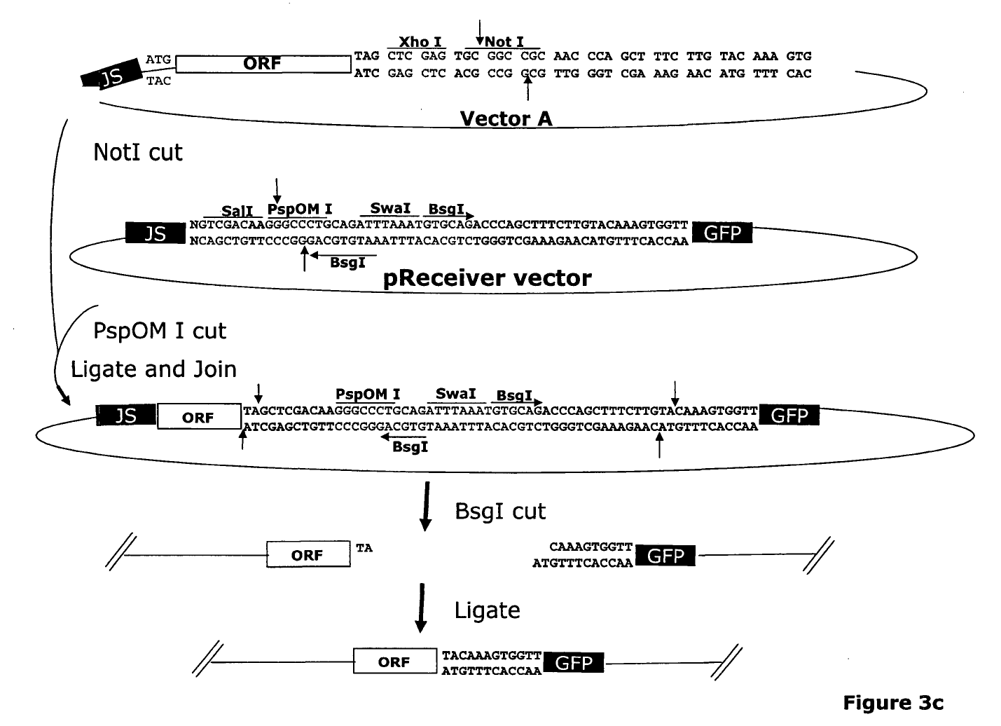

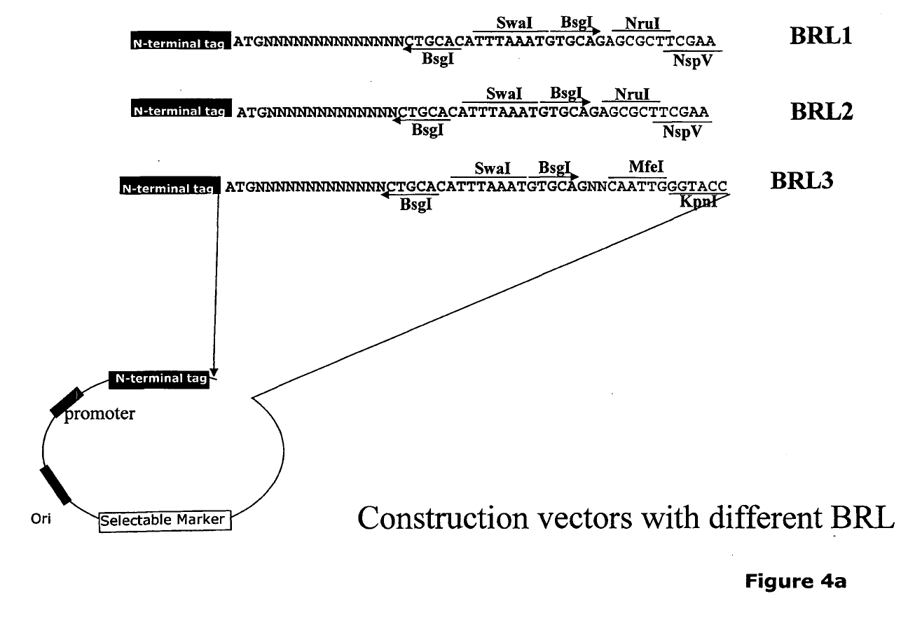

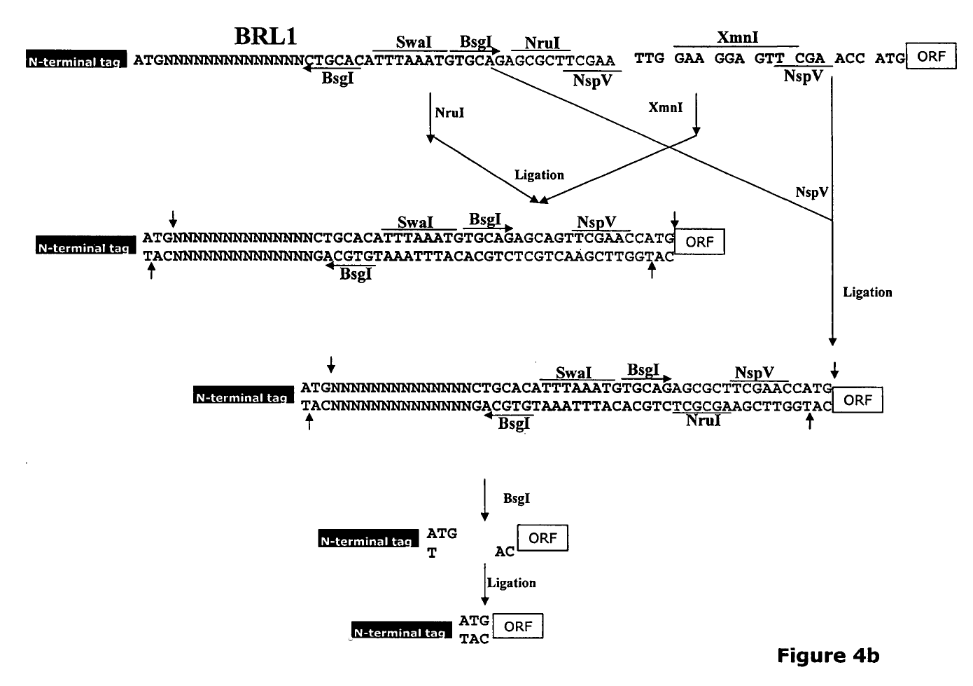

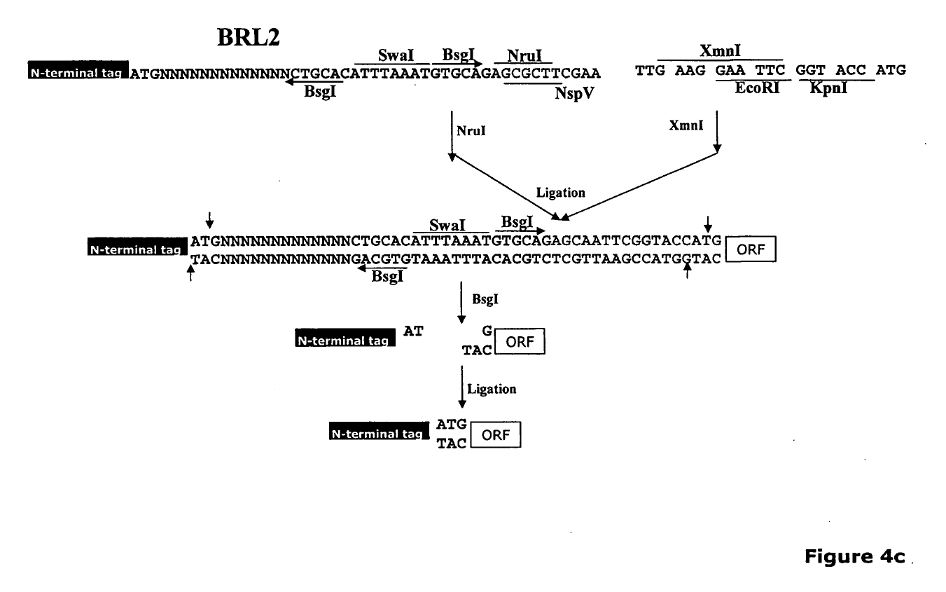

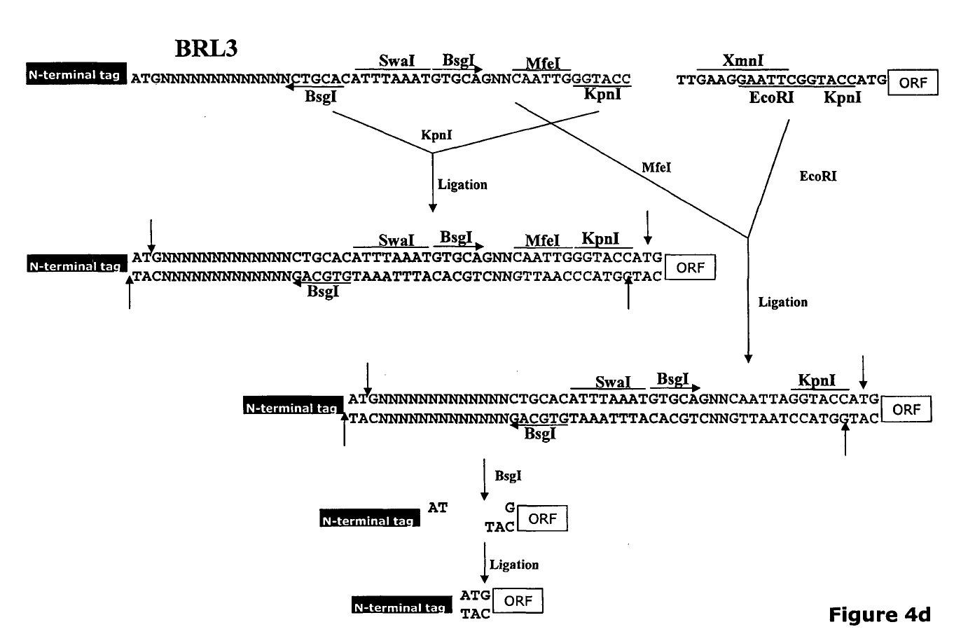

### EXAMPLES

### Example 1

### Construction of a C-terminal GFP Fusion Expression Vector With the Stop Codon Removed From an Existing ORF Clone With Stop Codon

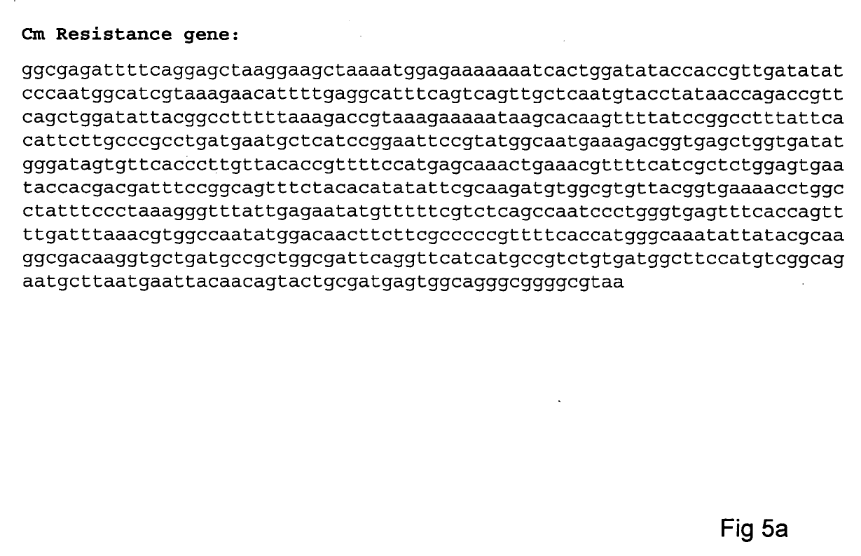

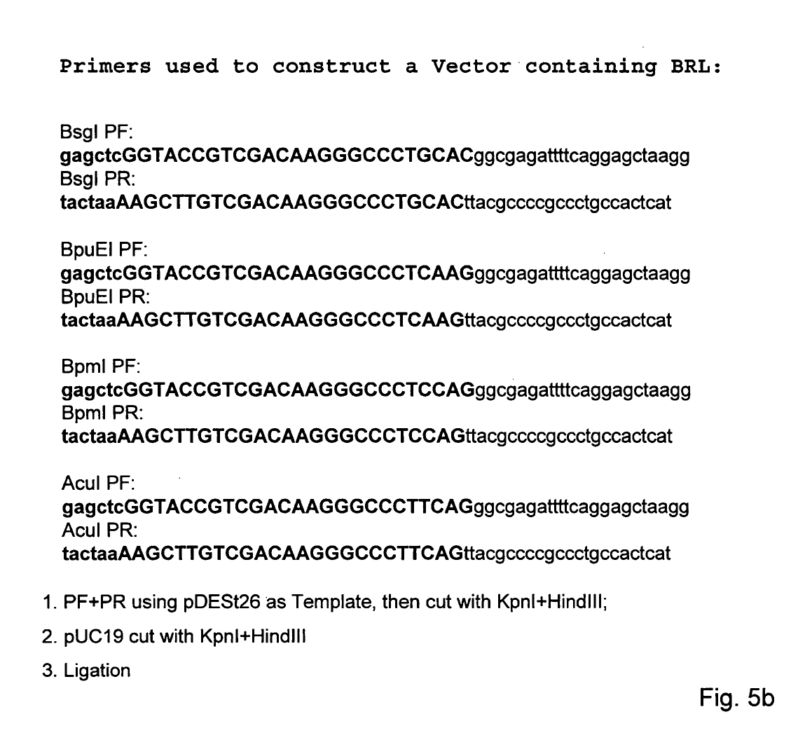

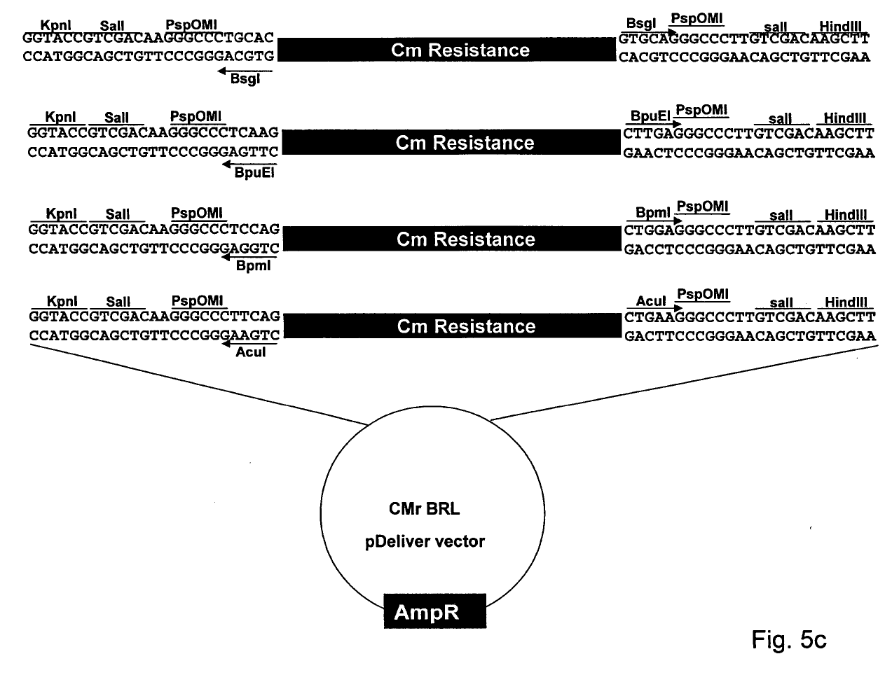

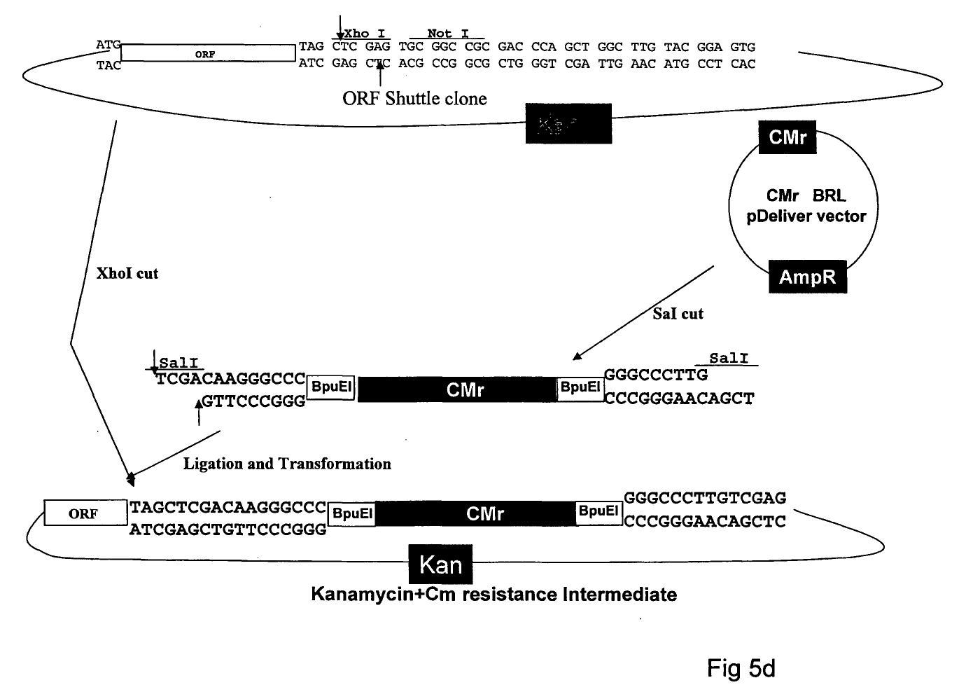

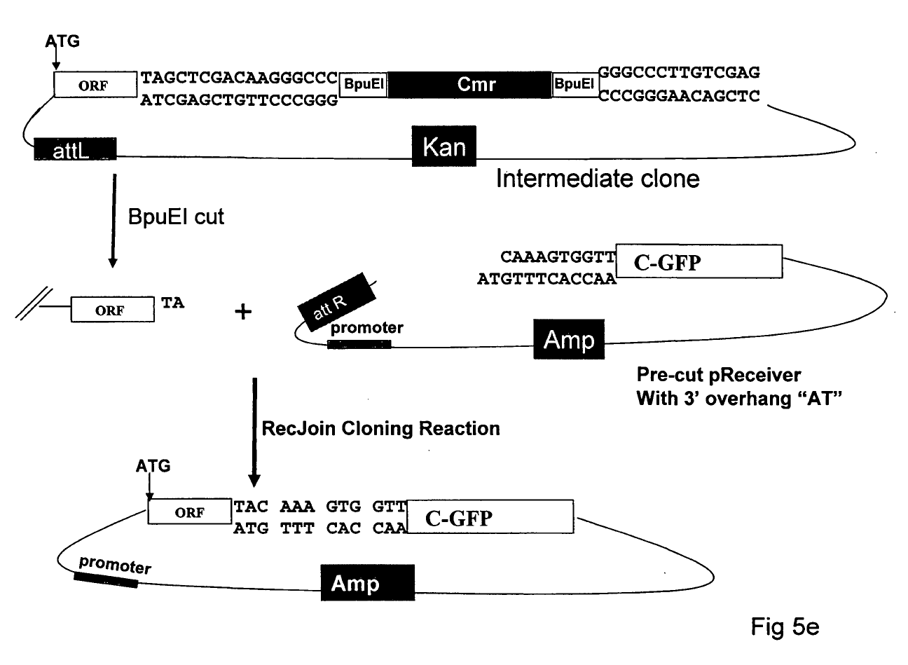

1. Construction of pDeliver (BRL) Vectors (as shown FIGS. 5a, 5b and 5c):

1) Synthesis 5 pairs primers as below:

BsgI FORWARD:
gagctcGGTACCGTCGACAAGGGCCCTGCACggcgagattttcaggagct
aagg
BsgI REVERSE:
tactaaAAGCTTGTCGACAAGGGCCCTGCACttacgccccgccctgccac
tcat
BpuEI FORWARD:
gagctcGGTACCGTCGACAAGGGCCCTCAAGggcgagattttcaggagct
aagg
BpuEI REVERSE:
tactaaAAGCTTGTCGACAAGGGCCCTCAAGttacgccccgccctgccac
tcat
BpmI FORWARD:
gagctcGGTACCGTCGACAAGGGCCCTCCAGggcgagattttcaggagct
aagg
BpmI REVERSE:
tactaaAAGCTTGTCGACAAGGGCCCTCCAGttacgccccgccctgccac
tcat
AcuI FORWARD:
gagctcGGTACCGTCGACAAGGGCCCTTCAGggcgagattttcaggagct
aagg
AcuI REVERSE:
tactaaAAGCTTGTCGACAAGGGCCCTTCAGttacgccccgccctgccac
tcat

2) Use the Forward and Reverse primer to amplify the Chloramphenicol resistance gene whose coding sequence is shown in FIG. 5a.
3) The amplification product and a pUC19 vector are cut with HindIII+KpnI;
4) Ligation and transformation, spread on LB plate contain 100 μg/ml Ampicillin and 12.5 μg/ml Chloramphenicol, then 37 [symbol] culture over night;
5) Pick clone, inoculate to LB medium containing 100 μg/ml Ampicillin and 12.5 μg/ml Chloramphenicol, then 37 [symbol] culture over night;
6) MiniPrep vector plasmid with QiaGen kit and then Sequencing the vector plasmid by ABI 3700

2. Preparation of the Intermediate Clone (as Shown in FIG. 5d)
1) ORF Express shuttle clone cut by XhoI or NotI;
2) pDeliver BRL vector cut by SalI or PspOMI;
3) Ligation and transformation, and spread on LB plate contain 60 ug/ml kanamycin and 12.5 ug/ml Chloramphenicol, then 37 [symbol] culture over night;
4) Pick and inoculate clone to LB medium containing 60 ug/ml Kanamycin ug/ml and 12.5 ug/ml Chloramphenicol, then 37 [symbol] culture over night;
5) MiniPrep intermediate clone plasmid with QiaGen kit

3. Remove Stop Codon of the Intermediate Clone and Construct the C-terminal GFP Expression Vector
1) Cut intermediate clone plasmid (about 500 ng plasmid DNA) by restriction enzyme type RE IIs (BsgI, BpuEI, or AcuI for corresponding intermediate clones, respectively), the stop codon was removed by the RE IIs, resulting in a 3′ overhang “TA” at the end of ORF in intermediate clone vector;
2) Ligation: add Pre-Cut pReceive vector with a 3′ overhang “AT” compatible with intermediate clone vector (as shown in FIG. 5e). The stop codon “TAG” was changed to “TAC”;
3) perform the RecJion cloning reaction at room temperature for 2 hrs;
4) Pick 3 μl Recjoin reaction produce, transfer into E. coli cell and using LB plate with Ampicillin.
5) MinPrep clone plasmid by Qiagen and confirm that the stop codon is removed by sequencing the plasmid with ABI 3700.

What is claimed is:

1. A method for modifying a first polynucleotide molecule that comprises a desired segment and extraneous nucleotides at either or both of the 5′ and 3′ ends of the desired segment, the method comprising
1) engineering the first polynucleotide molecule to contain an extraneous nucleotide removal linker (ENRL), or engineering a second polynucleotide molecule to contain an ENRL, wherein the ENRL comprises at least one recognition site for a Type IIs restriction endonuclease (RE IIs Site),
2) joining the first polynucleotide molecule with the second polynucleotide molecule to form a third polynucleotide molecule which comprises the desired segment, the extraneous nucleotides and at least one RE IIs site, and
3) digesting the third polynucleotide molecule with the RE IIs, wherein at least some of the extraneous nucleotides are removed.

2. The method of claim 1, wherein the first polynucleotide molecule comprises at its 3′ end a stop codon which is removed, or a 3′ non-translated sequences, or 5′ non-translated sequences which are removed.

3. The method of claim 1, wherein the first and second polynucleotide molecules comprise cognate sites that allow them to be joined via site-specific recombination.

4. The method of claim 1, wherein the first polynucleotide molecule is engineered to contain an ENRL using PCR with at least one primer that comprises the ENRL.

5. The method of claim 1, wherein the second polynucleotide molecule is a vector comprising an ENRL.

6. The method of claim 1, wherein the first and second polynucleotide molecules are joined by a combination of restriction digestion and ligation.

7. The method of claim 6, wherein the first and second polynucleotide molecules are joined by a combination of at least two of (1) site specific recombination, (2) restriction digestion, and (3) ligation.

8. The method of claim 1, wherein the third polynucleotide molecule is, concurrently or sequentially with the RE IIs digestion, digested with at least one Type II restriction endonuclease (RE II).

9. The method of claim 1, wherein the desired segment comprises an open reading frame (ORF), and digestion of the third polynucleotide molecule resulting in a fourth polynucleotide molecule which comprises the ORF.

10. The method of claim 9, wherein the fourth polynucleotide molecule is further circularized.

11. The method of claim 10, wherein the fourth polynucleotide molecule is further circularized via a ligation reaction.

12. The method of claim 9, wherein the fourth polynucleotide molecule comprises the ORF operably linked to at least one suitable transcription or translation regulatory element.

13. The method of claim 12, wherein the regulatory element is selected from the group of a promoter, an enhancer element, replication origin, and a ribosome binding site.

14. The method of claim 12, wherein the fourth polynucleotide molecule further comprises at least one of a selection marker, an expression tag, and a purification tag.

15. The method of claim 1, wherein the first polynucleotide molecule comprises a first selection marker, and the second polynucleotide molecule comprises a second selection marker, the third polynucleotide molecule comprises both the first and second selection markers.

16. The method according to claim 15, wherein said selectable marker is selected from the group consisting of antibiotic resistance gene, a fluorescent protein, an auxotrophic marker, a toxic marker and a phenotypic marker.

17. The method of claim 15, where the antibiotic resistance gene is selected from the group consisting of a chloramphenicol resistance gene, an ampicillin resistance gene, a tetracycline resistance gene, a Zeocin resistance gene, a spectinomycin resistance gene and a kanamycin resistance gene.

18. The method of claim 17, where the toxic marker is a gene selected from the group consisting of a ccdB gene, a gene encoding a tus protein, a kicB gene, a sacB gene, an ASK1 gene, a φX174 E gene and a DpnI gene.

19. The method of claim 15, wherein the third polynucleotide molecule is transformed into a suitable host cell and the host cell comprising the third polynucleotide molecule is selected based on both first and second selection markers.

20. The method of claim 15, wherein the third polynucleotide molecule comprises an open reading frame (ORF), and digestion with the RE IIs of the third polynucleotide molecule removes one of the two selection markers, and results in a fourth polynucleotide molecule comprising the ORF but only one of the first or second selection markers.

21. The method of claim 20, wherein the fourth polynucleotide molecule is further circularized.

22. The method of claim 21, wherein the fourth polynucleotide molecule is further circularized via a ligation reaction.

23. The method of claim 20, wherein the fourth polynucleotide molecule comprises the ORF operably linked to at least one suitable transcription or translation regulatory element.

24. The method of claim 20, wherein the fourth polynucleotide molecule comprises the ORF operably linked to at least one N-terminal Tag or a C-terminal Tag.

25. The method of claim 24, wherein the N-terminal or C-terminal tag is selected from the group of GST, HA, Myc, His6, Flag, SNAP, Avi, MBP, and Halo.

26. The method of claim 23, wherein the regulatory element is selected from the group consisting of a promoter, an enhancer element, replication origin, and a ribosomal biding site.

27. The method of claim 20, wherein the fourth polynucleotide molecule is transformed into a suitable host cell, and the host cell comprising the fourth polynucleotide molecule is selected based its characteristic of comprising only one of the first or second selection marker.

28. A vector comprising at least one ENRL.

29. A according to claim 28, further comprising a tag sequence downstream of the ENRL.

30. A according to claim 28, further comprising a tag sequence upstream of the ENRL.

31. A kit comprising 1) a vector of claims 29, and 2) at lease one Type IIs restriction enzyme.

32. The kit of claim 31, further comprises at least one type II restriction enzyme.

33. The kit of claim 32, further comprising at least one DNA ligase, and a suitable buffer for one or more of the enzymes.

34. A oligonucleotide comprises an ENRL either in its 5′-end or 3′-end, or both.
